(19) United States
(12) Patent Application Publication
(10) Pub. No.: US 2015/0217030 A1
(43) Pub. Date: Aug. 6, 2015

(54) METHOD FOR COATING A MEDICAL DEVICE WITH A CONFORMAL HYDROGEL

(71) Applicant: The Johns Hopkins University, Baltimore, MD (US)

(72) Inventors: Jason J. Benkoski, Ellicott City, MD (US); Peter V. Johnston, Baltimore, MD (US); Chao-Wei Hwang, West Friendship, MD (US); Gary Gerstenblith, Reisterstown, MD (US); Robert G. Weiss, Cockeysville, MD (US); Gordon Tomaselli, Lutherville, MD (US); Steven P. Schulman, Baltimore, MD (US); Jeffrey A. Brinker, Baltimore, MD (US)

(21) Appl. No.: 14/614,432

(22) Filed: Feb. 5, 2015

Related U.S. Application Data
(60) Provisional application No. 61/936,539, filed on Feb. 6, 2014.

(51) Int. Cl. A61L 31/10; A61L 31/14; A61L 31/16; B05D 1/18

(52) U.S. Cl. CPC A61L 31/10; B05D 1/18; A61L 31/145; A61L 31/16; A61L 2300/64; A61L 2420/02; A61L 2420/06; A61L 2300/606

(57) ABSTRACT

Certain embodiments according to the present invention provide a method for forming medical devices conformally coated with a hydrogel having a wide variety of therapeutic uses. In one aspect, certain embodiments of the invention provide a method for forming a hydrogel-coated medical device comprising immersing a medical device in a polymer solution to form an adhesive layer on an outer surface of the medical device and contacting the medical device with a hydrogel precursor solution having a pH of less than 7 to react the adhesive layer with the hydrogel precursor solution and form a conformal hydrogel coating.

### CROSS-REFERENCE TO RELATED APPLICATIONS

This application claims the benefit of U.S. Provisional Application No. 61/936,539 filed on Feb. 6, 2014, the entire contents of which are hereby incorporated herein by reference.

### TECHNICAL FIELD

The presently disclosed invention relates generally to hydrogel-coated medical devices and methods for forming the same.

### BACKGROUND

Cardiovascular disease afflicts more than 13 million Americans. Despite advances in heart failure therapy, there is no clinically available intervention to reverse underlying heart muscle injury. In recent years, stem cell therapy has been proposed as a way to regenerate the damaged tissue. It has become clear that stem cells' capacity to heal derives in large part from their ability to produce growth factors that accelerate the body's own repair mechanisms. However, current methods to administer stem cells to the heart, including intracoronary infusion and direct intramyocardial injection, are not conducive to the sustained production of beneficial growth factors. Rapid cell dilution, washout, and immune attack limit retention of viable stem cells, and, consequently, diminish the ability of the stem cells to produce sufficient growth factors to have desirable clinical effects.

Hydrogels have been proposed as a means for providing stem cells to damaged tissue. Hydrogels are water-insoluble polymers having the ability to swell in water or aqueous solution without dissolution and to retain a significant portion of water or aqueous solution within their structures. Hydrogels may possess a degree of flexibility similar to natural tissue, due to their significant water content. However, hydrogels are fragile and do not adhere well to most surfaces, let alone living tissue. Thus, they have only been used when injected into parts of the body outside of the circulatory system (e.g., subcutaneous injection).

One solution that has been proposed is to coat hydrogels on stents to be inserted into the circulatory system in order to overcome these issues associated with hydrogels. Stents have the capability to overcome any adhesion challenges by expanding against the inner wall of a blood vessel to secure it in place. However, uniformly coating a hydrogel on a medical device is difficult to achieve using standard polymerization methods. For example, it is difficult to achieve uniform polymerization using bulk polymerization because gelation often occurs before the solution can be mixed to full homogeneity. Additionally, radical polymerization or simple step growth polymerization tends to form a monolithic gel with an irregular shape in the absence of a mold. Dip-coating or spray-coating the polymer precursor onto a stent is also problematic because gravity draws excess polymer to the lower portion of the stent, and, more importantly, because the resulting hydrogel coating occludes the gaps between adjacent struts of the stent. The occlusion leads to lower surface area and the accompanying slower rate of protein permeation, but it is especially troublesome if the stent then blocks a branching blood vessel at the site of deployment. Furthermore, photopolymerization is problematic when used in conjunction with a stent because the stent will shadow the ultraviolet (UV) illumination. Polymerization will occur nonuniformly on the illuminated side, with little or no hydrogel polymer forming in the shadow. The UV light may also harm the cells embedded within the precursor solution in high doses. Although tube shapes may be possible using a mold or photomask, photocuring does not easily allow for openings between struts. Accordingly, photopolymerization using a mold carries the risk of possible occlusion similar to that seen with dip coating.

Therefore there at least remains a need in the art for a method for conformally coating a medical device with a hydrogel.

### BRIEF SUMMARY

One or more embodiments of the invention may address one or more of the aforementioned problems. Certain embodiments according to the present invention provide a method for forming medical devices conformally coated with a hydrogel having a wide variety of therapeutic uses. In one aspect, certain embodiments of the invention provide a method for forming a hydrogel-coated medical device. The method may comprise immersing a medical device in a polymer solution to form an adhesive layer on an outer surface of the medical device, optionally drying the medical device, contacting the medical device with a hydrogel precursor solution having a pH of less than 7 to react the adhesive layer with the hydrogel precursor solution and form a conformal hydrogel coating, and optionally rinsing away excess hydrogel precursor solution from the medical device to form a uniform conformal hydrogel coating. In such embodiments, the adhesive layer may comprise at least one polymer having at least one amine group, pH-modifying abilities, and reactivity with an activated ester.

In another aspect, the present invention provides a hydrogel-coated medical device. The hydrogel-coated medical device may comprise a medical device and a conformal hydrogel coating deposited on an outer surface of the medical device. In such embodiments, the conformal hydrogel coating may comprise a polyethylene glycol hydrogel, a plurality of stem cells, and Arg-Gly-Asp (RGD) oligopeptide adhesion molecules.

### BRIEF DESCRIPTION OF THE DRAWING(S)

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

FIG. 1 illustrates a side view of a hydrogel-coated medical device according to certain embodiments of the present invention.

FIG. 2 illustrates a side view of a hydrogel-coated medical device within a blood vessel according to certain embodiments of the present invention.

FIG. 3 illustrates a method of forming a hydrogel-coated medical device according to certain embodiments of the present invention showing optional steps of drying the medical device and rinsing away excess hydrogel precursor solution from the medical device to form a uniform conformal hydrogel coating.

FIG. 4 illustrates a method of forming a hydrogel-coated medical device according to certain embodiments of the present invention showing optional steps of drying the medical device and rinsing away excess hydrogel precursor solution from the medical device to form a uniform conformal hydrogel coating.

FIG. 5 illustrates the final solid fraction plotted against the initial solid fraction of a hydrogel according to certain embodiments of the present invention.

FIG. 6 illustrates the final organic solid fraction plotted against the molecular weight of a polyethylene glycol precursor according to certain embodiments of the present invention.

### DETAILED DESCRIPTION

The present invention now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present invention includes a method for forming a hydrogel-coated medical device. This method allows for a conformal coating of the medical device with a hydrogel in order to immobilize stem cells in the body. For instance, this method may provide, for example, a platform for stem cells to accomplish one or more of the following: release paracrine factors that promote regeneration in damaged tissue, prevent further tissue damage, and recruit endogenous stem cells to accelerate the healing process. As such, for example, the platform may be placed directly at or near the site of the injured tissue, or may be placed remotely from the injured tissue so long as the factors are enabled to communicate with the injured tissue.

Although stem cells are frequently referenced throughout this disclosure, stem cells serve only as an exemplary application of the present invention, which could be applicable to a wide variety of cell-based therapies. Moreover, although stents are frequently referenced as an application of the therapeutic sleeve device throughout this disclosure, stents serve only as an exemplary application of the present invention, which could be applicable to a wide variety of medical devices. Furthermore, although polyethylene glycol (PEG) is frequently referenced throughout this disclosure, polyethylene glycol serves only as an exemplary application of the present invention, which could be applicable to a wide variety of neutral water-soluble polymers that form hydrogels when crosslinked in water. Other examples of charged water-soluble polymers such as alginate can also apply to this invention. In those cases, pH-activation of the crosslinking reaction would be replaced by multi-valent cation-mediated cross-linking of the hydrogel through ionic bonds.

The terms “polymer” or “polymeric”, as used herein, may comprise homopolymers, copolymers, such as, for example, block, graft, random, and alternating copolymers, terpolymers, etc., and blends and modifications thereof. They may also comprise the various topologies of polymers that are possible, including infinite networks, branched, star, brush, and linear. Furthermore, unless otherwise specifically limited, the term “polymer” shall include all possible geometrical configurations of the material. These configurations include, but are not limited to, isotactic, syndiotactic, and atactic symmetries.

The term “step growth polymerization”, as used herein, may comprise a type of polymerization mechanism in which multi-functional monomers react to first form dimers, then trimers, and eventually long chain polymers, including infinite three-dimensional cross-linked polymer networks. Each chain end reacts with only one other chain end, leading to buildup of molecular mass between crosslinks. With high molecular mass between crosslinks, the mechanical strength, such as, e.g., tear resistance or the like, may be enhanced.

The term “cross-linked”, as used herein, may generally refer to a composition containing intermolecular cross-links and optionally intramolecular cross-links arising from the formation of covalent bonds, ionic bonds, hydrogen bonding, or any combination thereof “Cross-linkable” refers to a component or compound that is capable of undergoing reaction to form a cross-linked composition. A key property of a cross-linked polymer is that the bulk polymer does not melt or dissolve in any solvent. The cross-links retain the macroscopic shape of the bulk polymer even when the individual polymer chains would normally flow past each other. For example, linear polyethylene glycol dissolves in water, whereas cross-linked polyethylene glycol forms a hydrogel that absorbs water.

The term “pH-modifying”, as used herein, may generally refer to the ability of a compound to change the pH of an aqueous environment when the compound is placed in or dissolved in that environment.

The term “cation-modifying”, as used herein, may generally refer to the ability of a compound to change the salinity of an aqueous environment, particularly in reference to the concentration of multi-valent cations such as Ca2+ that form ionic crosslinks with negatively charged, water-soluble polymers such as sodium alginate or sodium hyaluranate.

As used herein, the term “layer” may comprise a region of a given material whose thickness is small compared to both its length and width. As used herein a layer need not be planar, for example, taking on the contours of an underlying substrate. A layer can be discontinuous (e.g., patterned). Terms such as “film,” “layer” and “coating” may be used interchangeably herein.

The term “tissue”, as used herein, may comprise any component of the body, including, but not limited to, muscle, blood vessels, bone, fat tissue, or skin.

The term “paracrine factor”, as used herein, may comprise one or more members of the entire secretome of a cell. As used herein, paracrine factors may act locally or systemically. Paracrine factors may comprise growth factors, nucleic acids (e.g., micro-RNA), or extracellular vesicles (e.g., exosomes).

The term “stem cell”, as used herein, may comprise hematopoietic or non-hematopoictic cells which exist in almost all tissues and have the capacity of self-renewal and the potential to differentiate into multiple cell types. Tissue injury is associated with the activation of immune/inflammatory cells, not only macrophages and neutrophils but also adaptive immune cells (e.g., CD4⁺ T cells, CD8⁺ T cells, B cells), which are recruited by factors from, for example, apoptotic cells, necrotic cells, damaged microvasculature and stroma. Meanwhile, inflammatory mediators (e.g., TNF-α, IL-1β, free radicals, chemokines, leukotrienes) are often produced by phagocytes in response to damaged cells and spilled cell contents. Thus, these inflammatory molecules and immune cells, together with endothelial cells and fibroblasts, orchestrate changes in the microenvironment that result in the mobilization and differentiation of stem cells into stroma and/or replacement of damaged tissue cells. Once stem cells have entered the microenvironment of injured tissues, for example, many factors (e.g., cytokines such as TNF-α, IL-1, IFN-γ, toxins of infectious agents and hypoxia) can stimulate the release of many factors from the stem cell secretome (e.g., epidermal growth factor (EGF), fibroblast growth factor (FGF), platelet-derived growth factor (PDGF), transforming growth factor β (TGF-β), vascular endothelial growth factor (VEGF), hepatocyte growth factor (HGF), insulin growth factor-1 (IGF-1), angiopoietin-1 (Ang-1), keratinocyte growth factor (KGF), stromal cell-derived factor-1 (SDF-1)). These growth factors, in turn, promote tissue regeneration and repair.

The term “adhesion molecule”, as used herein, may comprise proteins which are expressed on the surfaces of a variety of cell types and which mediate cell-cell interactions and subsequent cellular and biological responses, including, but not limited to, T cell activation, leukocyte transmigration, and inflammation. Adhesion molecules may comprise fibronectin, fibrinogen, laminins, collagen, vitronectin, proteoglycans, Arg-Gly-Asp (RGD) oligopeptides, and/or cell specific antibodies (e.g., anti-CD29 antibody).

The terms “hydrogel” and “hydrogel matrix system”, as used herein, may comprise a material which is not a readily flowable liquid and not a solid but a gel that contains a network of cross-linked polymer chains (“hydrogel polymers”) that are water-insoluble, sometimes found as a colloidal gel in which water is the dispersion medium. Hydrogels may contain a significant water content, absorbing at least 10 percent by weight of water when fully hydrated, due to formation interconnected polymer chains which bind to, entrap, absorb and/or otherwise hold water and thereby create a gel in combination with water, where water includes bound and unbound water. The term “hydrogel precursor solution”, as used herein, may comprise a flowable liquid solution containing a polymer that is capable of becoming crosslinked to form a solid hydrogel.

The term “medical device”, as used herein, may comprise any medical device capable of being inserted into a mammalian (e.g., human) body and used in conjunction with a conformal hydrogel coating. Medical devices may comprise stents, stent sleeves, pacemakers, vascular grafts, implantable cardioverter-defibrillators, pacemaker electrodes, implantable cardioverter-defibrillator leads, biventricular implantable cardioverter-defibrillator leads, artificial hearts, artificial valves, ventricular assist devices, balloon pumps, catheters, central venous lines, implants, or sensors. Blood clots are one potential problem with inserting these medical devices, but the devices may be coated with some additive or component that reduces the risk of blood clots. The terms “antifouling” and “anti-clotting”, as used herein, may generally refer to the ability to prevent accumulations (e.g., blood clots) from forming on medical devices.

I. Method for Forming a Hydrogel-Coated Medical Device

In one aspect, the present invention provides a method for forming medical devices conformally coated with a hydrogel having a wide variety of therapeutic uses. For instance, this method may provide, for example, a platform for stem cells to accomplish one or more of the following: release paracrine factors that promote regeneration in damaged tissue, prevent further tissue damage, and recruit endogenous stem cells to accelerate the healing process. In general, methods for forming hydrogel-coated medical devices according to certain embodiments of the present invention may include immersing a medical device in a polymer solution to form an adhesive layer on an outer surface of the medical device, optionally drying the medical device, contacting the medical device with a hydrogel precursor solution having a pH of less than 7 to react the adhesive layer with the hydrogel precursor solution and form a conformal hydrogel coating, and optionally rinsing away excess hydrogel precursor solution from the medical device to form a uniform conformal hydrogel coating. In certain embodiments, the adhesive layer may comprise at least one polymer having at least one amine group, pH-modifying abilities, and reactivity with an activated ester.

In accordance with certain embodiments of the present invention, the adhesive layer may comprise at least one of a poly(allylamine), a polylysine, or a polyethylenimine. The adhesion promoter must meet two conditions: (i) it must contain pendant functional groups that can react with pendant functional groups of a water soluble polymer (i.e., hydrogel precursor) to form crosslinks, and (ii) it must contain pendant functional groups or a leaching chemical that change the conditions of the local aqueous medium so as to activate or increase the rate of reaction between the functional pendant groups of the adhesion promoter and the hydrogel precursor, as well as the cross-linking reactions taking place within the hydrogel precursor solution in close proximity to the surface. Primarily, the latter is achieved by changing the local pH near the surface in the case of covalent bond formation, or by leaching multi-valent cations into the adjacent hydrogel precursor solution in the case of ionic bond formation. In further embodiments, for example, the adhesive layer may comprise a poly(allylamine) generalized by the structure represented by Formula 1:

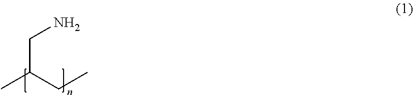

In other embodiments, for instance, the adhesion promoter may consist of a combination of poly(4-vinyl pyridine) and poly(vinyl alcohol). If, instead of covalent cross-links, an anionic hydrogel such as sodium alginate is used, and it is held together by ionic crosslinks, then the adhesion promoter will generally again be a multi-valent polycation, such as poly(allylamine), polyethylenimine, poly(lysine), or poly(dimethyldiallylammonium chloride). To further promote the cross-linking reaction within the anionic hydrogel that forms a conformal coating on the surface, the polycation may be spiked with the salt of a multi-valent cation, such as CaCl₂. that will then leach into the aqueous medium and increase the local concentration of Ca²⁺ near the surface.

In accordance with certain embodiments of the present invention, the medical device may comprise any insertable medical device. In such embodiments, the medical device may comprise a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor. In certain embodiments, for example, the medical device may comprise a stent. According to certain embodiments, for instance, the medical device may comprise a stainless steel stent. In such embodiments, the adhesive layer on the stainless steel stent may comprise a poly(allylamine) according to Formula 1. In further embodiments, for example, the medical device may comprise a pacemaker electrode. In such embodiments, the adhesive layer on the pacemaker electrode may comprise a layer of poly(allylamine) deposited on silicone.

In accordance with certain embodiments of the present invention, the hydrogel precursor solution may be formed by mixing a first neutral water-soluble polymer that forms hydrogels when crosslinked in water with at least three activated ester groups and mixing a second neutral water-soluble polymer that forms hydrogels when crosslinked in water with at least two amine groups to form a hydrogel polymer network. According to certain embodiments, for example, the first, second, or both neutral water-soluble polymers that form hydrogels when crosslinked in water may comprise polyethylene glycol (PEG).

In accordance with certain embodiments of the present invention, the hydrogel precursor solution may alternatively consist of a negatively charged polyelectrolyte such as sodium alginate and a positively charged cross-linking agent such as Ca²⁺. The polyelectrolyte must have at least three negatively charged pendant groups and the cation must have a valency of at least 2. Sodium alginate and Ca²⁺ is the most common example. Typically, one dissolves CaCl₂ in water to initiate the formation of ionic cross-links within the sodium alginate aqueous solution. Once mixed together, the Ca²⁺ ions coordinate ionically with at least two carboxylate groups on the sodium alginate. Since sodium alginate has well more than three negatively charged carboxylate groups, the formation of an infinite cross-linked network is assured.

As used herein, “average functionality” may generally refer to the number of covalent or ionic bonds that may be formed by the active agent with a corresponding reactive compound. Use of active agents with a functionality of greater than 2 may therefore define a monomer that is capable of branching and/or crosslinking. In some embodiments, for instance, the hydrogel polymer network may comprise an average functionality of from about 3 to about 10. In further embodiments, for example, the hydrogel polymer network may comprise an average functionality of from about 3 to about 7. In other embodiments, for instance, the hydrogel polymer network may comprise an average functionality of about 5. As such, in certain embodiments, the hydrogel polymer network may comprise an average functionality of at least about any of the following: 3, 4, and 5 and/or at most about 10, 7, and 5 (e.g., about 3-7, about 4-5, etc.).

According to certain embodiments, contacting the coated medical device with the hydrogel precursor solution may comprise step-growth polymerizations. In such embodiments, the step-growth polymerizations may comprise reacting PEG with one of N-hydroxysuccinimide (NHS) ester/amine, isocyanate/amine, epoxy/amine, isothiocyanate/amine, alcohol/glutamate, thiol/maleimide, isocyanate/alcohol, or isocyanate/polyol reaction chemistries. In other embodiments, for instance, the step-growth polymerizations may comprise reacting PEG with NHS ester/amine, which are illustrated by the reactant structures represented by Formula 2:

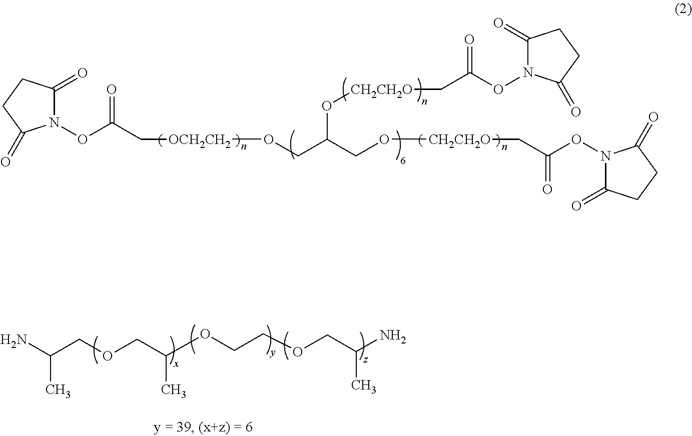

In such embodiments, for example, crosslinks form through the reaction between NHS-activated esters and amine groups. In such embodiments, for instance, the two functional groups react to form an amide bond with the loss of the NHS group. This reaction occurs at the greatest rate at a pH between 7 and 10 but is much slower at a pH below 6. As such, in certain embodiments, the hydrogel precursor solution may have a pH of about 6 or lower. In such embodiments, for example, the hydrogel precursor solution may have a pH from about 4 to about 6. In further embodiments, for instance, the hydrogel precursor solution may have a pH from about 4.5 to about 6. In other embodiments, for example, the hydrogel precursor solution may have a pH from about 5 to about 6. As such, in certain embodiments, the hydrogel precursor solution may have a pH from at least about any of the following: 4, 4.5, and 5 and/or at most about 6, 5.5, and 5 (e.g., about 4.5-6, about 5-5.5, etc.).

In further embodiments, for example, the step-growth polymerizations may comprise reacting PEG with isocyanate/alcohol or isocyanate/polyol. In such embodiments, for instance, the method may further comprise embedding a non-toxic catalyst in the hydrogel precursor solution. In certain embodiments, for example, the non-toxic catalyst may comprise a 1,4-diazabicyclo[2.2.2]octane catalyst (DABCO® from Air Products and Chemicals, Inc., 7201 Hamilton Blvd., Allentown, Pa. 18195), 1,8-diazabicycloundec-7-ene (DBU) catalyst, or other non-toxic tertiary amine catalysts.

According to certain embodiments, contacting the coated medical device with the hydrogel precursor solution may comprise ionic crosslinking. In such embodiments, the ionic crosslinging may comprise mixing a negatively charged polyelectrolyte such as sodium alginate, heparin, poly(styrene sulfonate), poly(acrylic acid), poly(methacrylic acid), sodium hyaluronate or similar. In other embodiments, for instance, the ionic crosslinking may comprise mixing sodium alginate with CaCl₂, which are illustrated by the reactant structures represented by Formula 3:

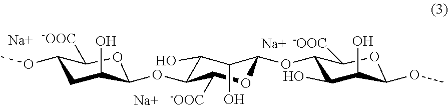

In such embodiments, for example, crosslinks form through the ionic coordination of Ca²⁺ with multiple carboxylate groups. This reaction occurs at the greatest rate above a pH of 3.5.

According to certain embodiments of the present invention, for example, the hydrogel precursor solution may comprise from about 5 wt. % to about 50 wt. % organic solids (e.g., polymers). In further embodiments, for instance, the hydrogel precursor solution may comprise from about 10 wt. % to about 30 wt. % organic solids (e.g., polymers). In other embodiments, for example, the hydrogel precursor solution may comprise about 25 wt. % organic solids (e.g., polymers). As such, in certain embodiments, the hydrogel precursor solution may comprise an organic solid (e.g., polymer) weight percent from at least about any of the following: 5, 10, 20, and 25 wt. % and/or at most about 50, 30, 28, and 25 wt. % (e.g., about 5-30 wt. %, about 20-25 wt. %, etc.). In such embodiments, for example, higher initial solid fractions may facilitate more efficient crosslinking reactions between the NHS-PEG and the diamine-PEG or between sodium alginate and Ca²⁺.

According to certain embodiments of the present invention, after the hydrogel crosslinking is formed, it may expand many times its own weight (e.g., 4×-5×) by soaking up additional water. In such embodiments, water will continue to absorb until the crosslinked hydrogel reaches equilibrium. In certain embodiments, for example, equilibrium may occur when the crosslinked hydrogel comprises about 1-50 wt. % organic solids (e.g., polymers). In further embodiments, for instance, equilibrium may occur when the crosslinked hydrogel comprises about 3-30 wt. % organic solids (e.g., polymers). In other embodiments, for example, equilibrium may occur when the crosslinked hydrogel comprises about 5-10 wt. % organic solids (e.g., polymers). As such, in certain embodiments, the crosslinked hydrogel may comprise an organic solid (e.g., polymer) weight percent from at least about any of the following: 1, 3, and 5 wt. % and/or at most about 50, 30, and 10 wt. % (e.g., about 3-10 wt. %, about 5-10 wt. %, etc.).

In accordance with certain embodiments of the present invention, for instance, the hydrogel precursor solution may comprise organic solids (e.g., polymers) at a molecular weight from about 1000 g/mol to about 100,000 g/mol. In further embodiments, for example, the hydrogel precursor solution may comprise organic solids (e.g., polymers) at a molecular weight from about 1500 g/mol to about 50,000 g/mol. In other embodiments, for instance, the hydrogel precursor solution may comprise organic solids (e.g., polymers) at a molecular weight from about 2000 g/mol to about 40,000 g/mol. As such, in certain embodiments, the hydrogel precursor solution may comprise organic solids (e.g., polymers) at a molecular weight from at least about any of the following: 1000, 1500, and 2000 g/mol and/or at most about 100,000, 50,000, and 40,000 g/mol (e.g., about 1000-40,000 g/mol, about 2000-40,000 g/mol, etc.). In such embodiments, high molecular weight may lead to lower crosslink densities and lower stiffness.

In the above embodiments, the equilibrium fraction of organic solids in a hydrogel increases with increasing crosslink density and decreases with the molecular weight of the polyethylene glycol between crosslinks according to Formula 3:

$${ρ}_{x} = - \frac{1}{v} ( \frac{ln ( 1 - {v}_{p} ) + {v}_{p} + χ {v}_{p}^{2}}{{v}_{p}^{1 / 3} - {v}_{p}^{2} / 2} ) \\ and \\ {ρ}_{x} = \frac{2}{{f}_{av}} \frac{ρ}{{M}_{c}} \qquad ( 4 )$$

where υₚ is the equilibrium fraction of organic solids in a hydrogel, ρₓ is the density as determined by the number of crosslinks per volume in mol/L, ν̅ is the molar volume of water, χ is the Flory-Huggins chi parameter, ƒₐᵥ is the average functionality of the monomers, and M_{c} is the molecular weight of the of the polymer chains between crosslinks.

In accordance with certain embodiments of the present invention, the hydrogel precursor solution may comprise a plurality of stem cells. In some embodiments, for example, the plurality of stem cells may comprise mesenchymal stem cells. In further embodiments, for instance, the mesenchymal stem cells may comprise human mesenchymal stem cells. In certain embodiments, the stem cells may be mixed into the hydrogel precursor solution to embed the stem cells in the hydrogel-coated medical device. In such embodiments, the stem cells may be mixed into the hydrogel precursor solution before immersing the medical device in the hydrogel precursor solution so that the hydrogel precursor solution is still in a viscous liquid state when mixed. According to some embodiments, for instance, the hydrogel precursor solution may comprise a concentration of stem cells from about 1 million cells/mL to about 10 million cells/mL. In further embodiments, for example, the hydrogel precursor solution may comprise a concentration of stem cells from about 5 million cells/mL to about 8 million cells/mL. In other embodiments, for instance, the hydrogel precursor solution may comprise a concentration of stem cells of about 8 million cells/mL. As such, in certain embodiments, the hydrogel precursor solution may comprise a concentration of stem cells of at least about any of the following: 1 million, 5 million, and 8 million cells/mL and/or at most about 10 million, 9 million, and 8 million cells/mL (e.g., about 5 million-9 million cells/mL, about 5 million-8 million cells/mL, etc.).

In such embodiments, the hydrogel precursor solution may comprise Arg-Gly-Asp (RGD) oligopeptide adhesion molecules. In some embodiments, for example, the hydrogel precursor solution may comprise linear RGD oligopeptide adhesion molecules. In other embodiments, for instance, the hydrogel precursor solution may comprise cyclic RGD oligopeptide adhesion molecules. Cyclic RGD oligopeptide adhesion molecules may confer greater stability and selectivity over linear RGD oligopeptide adhesion molecules. In certain embodiments, for example, the hydrogel precursor solution may comprise cyclo(Arg-Gly-Asp-d-Phe-Lys), illustrated by the structure represented by Formula 4:

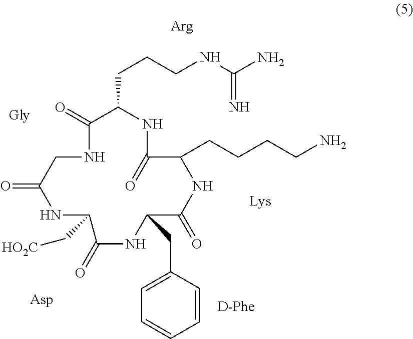 [SMILES: N=C(N)NCCC[C@@H]1NC(=O)C(CCCCN)NC(=O)[C@H](Cc2ccccc2)NC(=O)[C@@H](CC(=O)O)NC(=O)CNC1=O]

In such embodiments, for instance, the free amine group on the additional lysine residue may react with NHS-PEG to become incorporated into the hydrogel. The free amine group may similarly incorporate into alginate hydrogels through the same activated NHS-ester chemistry. According to certain embodiments, for example, the hydrogel precursor solution may comprise from about 1 to about 20 mM of RGD oligopeptide adhesion molecules to promote stem cell adhesion to the hydrogel. In such embodiments, for instance, the addition of the RGD oligopeptide adhesion molecules may permit the stem cells to pull themselves into a state of tension, and, as a result, lens-like shapes, to form focal adhesion sites with the hydrogel. As such, the addition of RGD oligopeptide adhesion molecules may facilitate the formation of focal adhesion points through interactions between the RGD oligopeptide adhesion molecules and integrin, and cell viability may be improved.

In accordance with certain embodiments of the present invention, the adhesive layer may adjust the pH of the hydrogel precursor solution on contact with the medical device to at least about 7.4. In such embodiments, for example, the adhesive layer may adjust the pH of the hydrogel precursor solution on contact with the medical device to about 7-8. In further embodiments, for instance, the adhesive layer may adjust the pH of the hydrogel precursor solution on contact with the medical device to about 7.4-7.8. In other embodiments, for example, the adhesive layer may adjust the pH of the hydrogel precursor solution on contact with the medical device to about 7.4-7.6.

In accordance with certain embodiments of the present invention, the adhesive layer may leach Ca²⁺ into the aqueous hydrogel precursor solution adjacent to the surface. The local concentration of Ca²⁺ will transiently increase then decrease over time, but the final concentration will typically range from about 0.1 to 10 mM in the solid alginate hydrogel.

In certain embodiments, for example, by immersing a poly(allylamine)-coated medical device in the hydrogel precursor solution, the poly(allylamine) may raise the pH near the surface in order to initiate polymerization, which may only occur at pH>7. In such embodiments, a basic polycation has been applied to the surface of the device via poly(allylamine). According to certain embodiments, the hydrogel precursor solution containing the stem cells may be applied to the medical device, for example, via painting, spraying, dip-coating, or immersion. When hydrated, the polymer in the hydrogel precursor solution generates OH-ions that diffuse away from the surface, which initializes the pH change that uniformly grows according to Formula 5:

ΔpH=(Dt)^{1/2} (6)

where D is the diffusion coefficient, and t is time. As such, the hydrogel thickness grows with the square root of time. Thus, the hydrogel thickness may be determined by controlling the time that the poly(allylamine)-coated medical device is soaked in the hydrogel precursor solution. According to certain embodiments, the polymerization rate may be increased by applying a thicker film of poly(allylamine) to the medical device. When the hydrogel coating has grown to a desired thickness, the excess hydrogel precursor solution may be rinsed away from the medical device. In some embodiments, for example, the hydrogel precursor solution may be rinsed away from the medical device with phosphate buffered saline (PBS).

In certain embodiments, for example, by immersing a poly(allylamine)-coated medical device that is spiked with CaCl₂ in the hydrogel precursor solution, the CaCl₂ may raise the Ca²⁺ concentration near the surface in order to initiate polymerization, which will occur at essentially any concentration. In such embodiments, a polycation loaded with a water-soluble salt that has a multi-valent cation has been applied to the surface of the device via poly(allylamine). According to certain embodiments, the hydrogel precursor solution containing the stem cells may be applied to the medical device, for example, via painting, spraying, dip-coating, or immersion. When hydrated, the polymer in the hydrogel precursor solution leaches Ca²⁺ ions that diffuse away from the surface, which initializes the Ca²⁺ concentration front that uniformly grows in thickness according to Formula 5 above. The hydrogel thickness again grows with the square root of time. Thus, the hydrogel thickness may be determined by controlling the time that the poly(allylamine)-coated medical device is soaked in the hydrogel precursor solution. According to certain embodiments, the polymerization rate may be increased by applying a thicker film of poly(allylamine) to the medical device or by increasing the loading of CaCl₂. When the hydrogel coating has grown to a desired thickness, the excess hydrogel precursor solution may be rinsed away from the medical device. In some embodiments, for example, the hydrogel precursor solution may be rinsed away from the medical device with phosphate buffered saline (PBS).

As such, according to certain embodiments of the present invention, the conformal hydrogel coating may form an anti-fouling surface on the medical device. In such embodiments, the conformal hydrogel coating may immobilize stem cells located therein while permitting permeation of nutrients, waste, and paracrine factors to promote regeneration at a damage site, prevent further tissue damage, and recruit endogenous stem cells to accelerate the healing process.

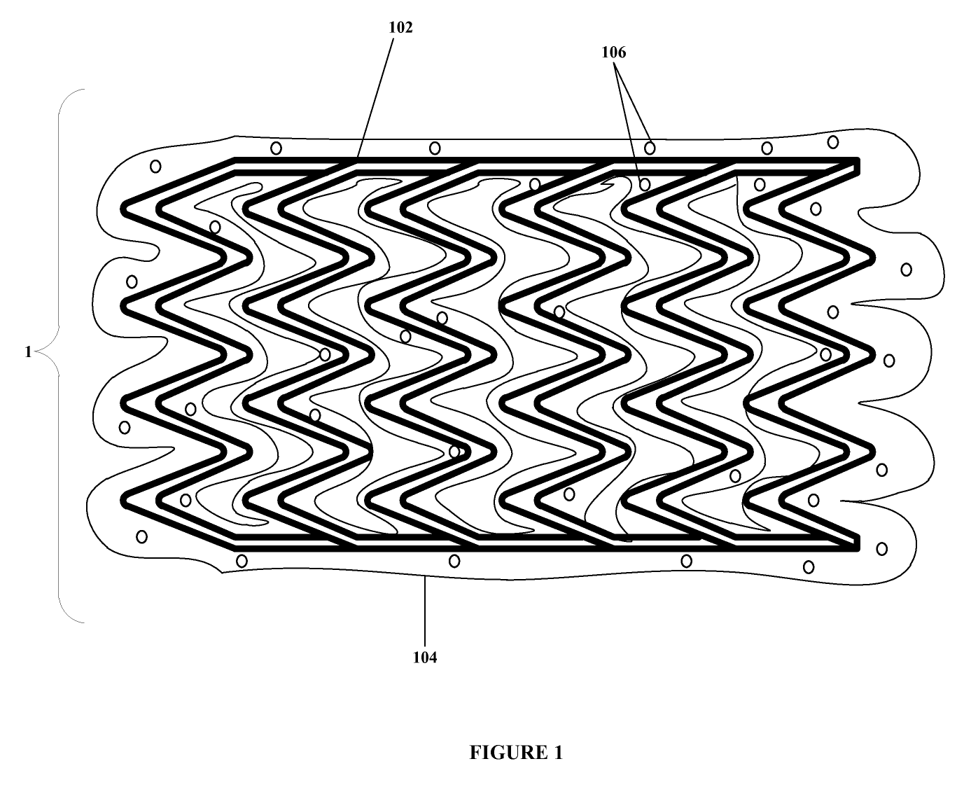

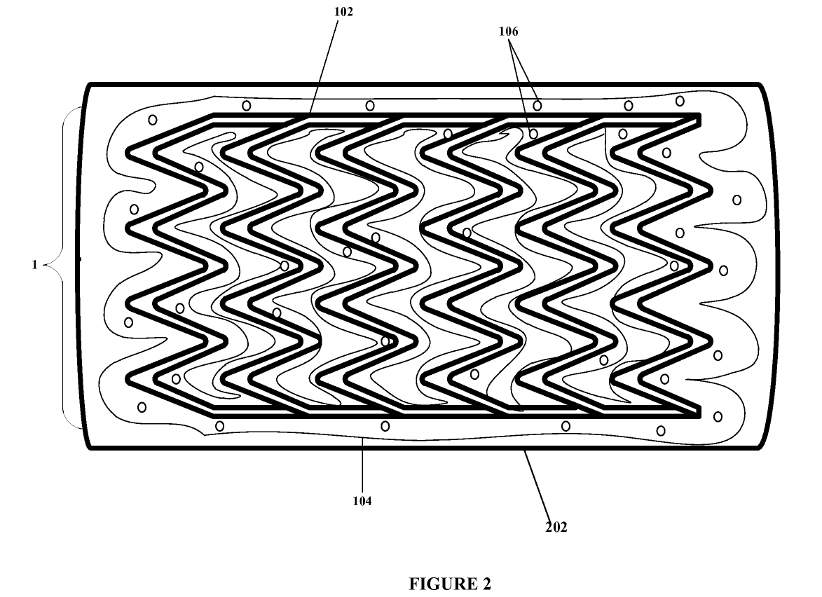

FIG. 1, for example, illustrates a side view of a hydrogel-coated medical device according to certain embodiments of the present invention. As shown in FIG. 1, the hydrogel-coated medical device 1 illustrated in FIG. 1 includes a stent 102 conformally coated with a hydrogel 104. Stem cells 106 are embedded in the hydrogel 104. FIG. 2, for example, illustrates a side view of a hydrogel-coated medical device inside a blood vessel according to certain embodiments of the present invention. Specifically, as shown in FIG. 2, the hydrogel coated medical device 1 illustrated in both FIGS. 1 and 2 includes a stent 102 conformally coated with a hydrogel 104. Stem cells 106 are embedded in the hydrogel 104. The entire hydrogel-coated medical device 1 is shown inside a blood vessel 202.

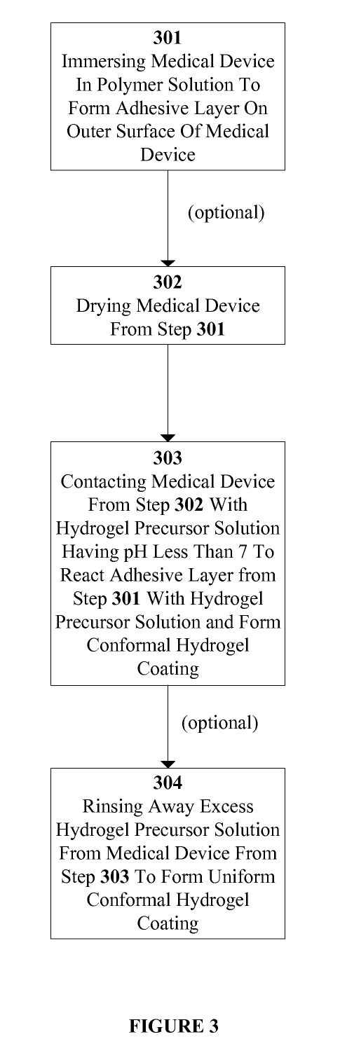

FIG. 3, for example, illustrates a method of forming a hydrogel-coated medical device according to certain embodiments of the present invention showing optional steps of drying the medical device and rinsing away excess hydrogel precursor solution from the medical device to form a uniform conformal hydrogel coating. As shown in FIG. 3, the method comprises immersing a medical device in a polymer solution to form an adhesive layer on an outer surface of the medical device in step 301 and optionally drying the medical device from step 301 in step 302. The method further comprises step 303, which comprises contacting the medical device from step 302 with a hydrogel precursor solution having a pH less than 7 to react the adhesive layer from step 301 with the hydrogel precursor solution and form a conformal hydrogel coating. The process further comprises optionally rinsing away excess hydrogel precursor solution from the medical device from step 303 to form a uniform conformal hydrogel coating in step 304.

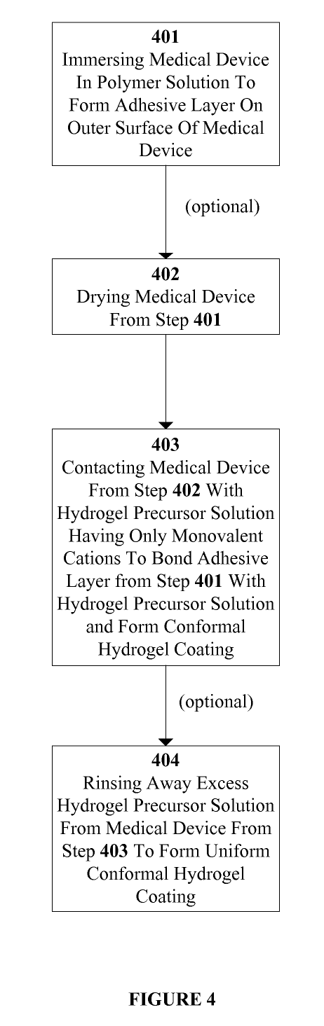

FIG. 4, for example, illustrates a method of forming a hydrogel-coated medical device according to certain embodiments of the present invention showing optional steps of drying the medical device and rinsing away excess hydrogel precursor solution from the medical device to form a uniform conformal hydrogel coating. As shown in FIG. 4, the method comprises immersing a medical device in a polymer solution to form an adhesive layer on an outer surface of the medical device in step 401 and optionally drying the medical device from step 401 in step 402. The method further comprises step 403, which comprises contacting the medical device from step 402 with a hydrogel precursor solution having only monovalent cations to bond the adhesive layer from step 401 with the hydrogel precursor solution and form a conformal hydrogel coating. The process further comprises optionally rinsing away excess hydrogel precursor solution from the medical device from step 403 to form a uniform conformal hydrogel coating in step 404.

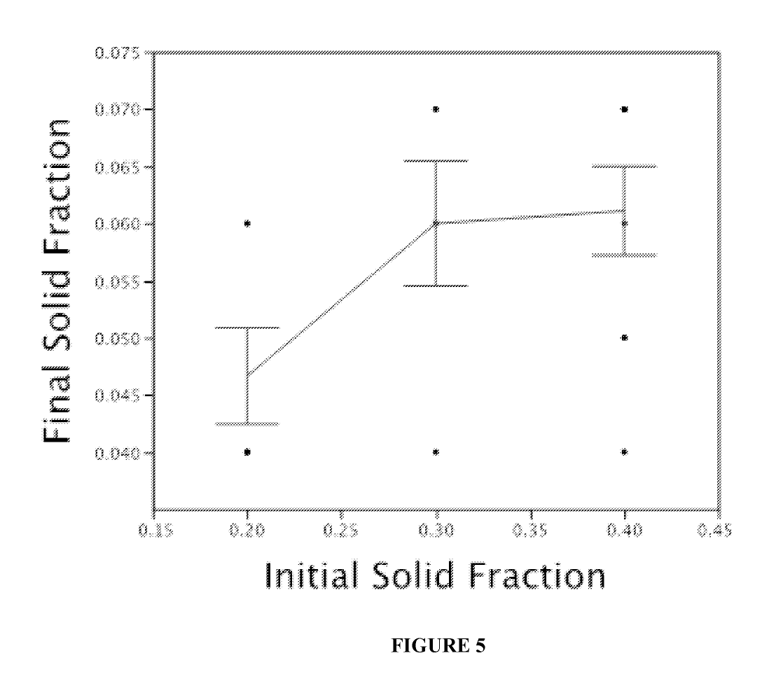

FIG. 5, for example, illustrates the final solid fraction plotted against the initial solid fraction of a hydrogel according to certain embodiments of the present invention. As shown in FIG. 4, greater reaction efficiency can be seen in the fact that the final solid fraction is higher with increasing initial solid fraction.

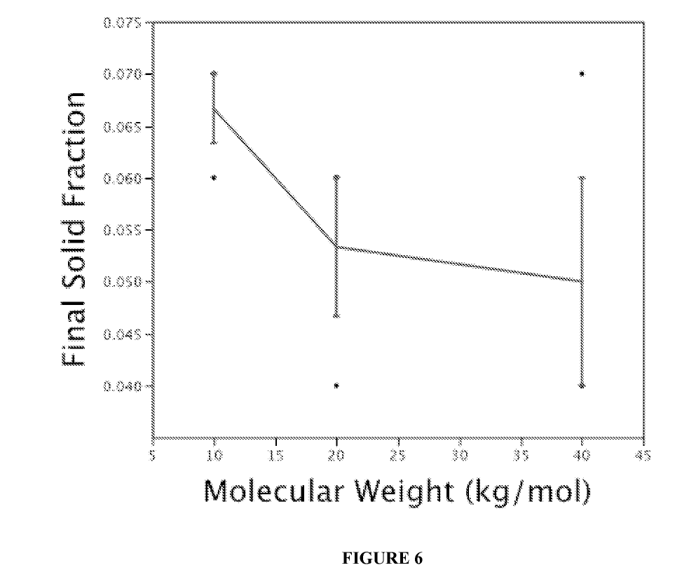

FIG. 6, for example, illustrates the final organic solid fraction plotted against the molecular weight of a PEG precursor according to certain embodiments of the present invention. As shown in FIG. 5, the equilibrium fraction of organic solids in a hydrogel decreases with the molecular weight of the PEG between crosslinks.

II. Hydrogel-Coated Medical Device

In another aspect, the present invention provides a hydrogel-coated medical device having a wide variety of therapeutic uses. For instance, the hydrogel-coated medical device may provide, for example, a platform for stem cells to accomplish one or more of the following: release paracrine factors that promote regeneration in damaged tissue, prevent further tissue damage, and recruit endogenous stem cells to accelerate the healing process. In general, hydrogel-coated medical devices according to certain embodiments of the present invention may include a medical device and a conformal hydrogel coating deposited on an outer surface of the medical device, in which the conformal hydrogel coating may comprise a PEG hydrogel, a plurality of stem cells, and RGD oligopeptide adhesion molecules. In accordance with certain embodiments of the present invention, the conformal hydrogel coating may immobilize the stem cells in the hydrogel but permit permeation of nutrients, waste, and growth factors.

In accordance with certain embodiments of the present invention, the medical device may comprise any insertable medical device. In such embodiments, the medical device may comprise a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor. In certain embodiments, for example, the medical device may comprise a stent. According to certain embodiments, for instance, the medical device may comprise a stainless steel stent. In further embodiments, for example, the medical device may comprise a pacemaker electrode.

In accordance with certain embodiments of the present invention, the hydrogel-coated medical device may be formed by immersing a medical device in a polymer solution to form an adhesive layer on an outer surface of the medical device, optionally drying the medical device, contacting the medical device with a hydrogel precursor solution having a pH of less than 7 to react the adhesive layer with the hydrogel precursor solution and form a conformal hydrogel coating, and optionally rinsing away excess hydrogel precursor solution from the medical device to form a uniform conformal hydrogel coating. In such embodiments, the adhesive layer may comprise at least one polymer having at least one amine group, pH-modifying abilities, and reactivity with an activated ester. As such, the hydrogel-coated medical device may be formed according to any of the embodiments disclosed in regard to the method for forming a hydrogel-coated medical device.

In accordance with certain embodiments of the present invention, the hydrogel-coated medical device may alternatively be formed by immersing a medical device in a polycation solution to form an adhesive layer on an outer surface of the medical device, optionally drying the medical device, contacting the medical device with an anionic hydrogel precursor solution having only monovalent salts to form ionic bonds between the polycation and anionic hydrogel and to then subsequently form ionic bonds with freely diffusing multivalent cations to form a conformal hydrogel coating, and optionally rinsing away excess hydrogel precursor solution from the medical device to form a uniform conformal hydrogel coating. In such embodiments, the adhesive layer may comprise at least one polymer having at least three negatively charged pendant groups, and a water-soluble multi-valent cation that can leach from the surface. As such, the hydrogel-coated medical device may be formed according to any of the embodiments disclosed in regard to the method for forming a hydrogel-coated medical device.

FIG. 1, as previously discussed, illustrates a side view of a hydrogel-coated medical device according to certain embodiments of the present invention. As shown in FIG. 1, the hydrogel-coated medical device 1 illustrated in FIG. 1 includes a stent 102 conformally coated with a hydrogel 104. Stem cells 106 are embedded in the hydrogel 104.

### EXAMPLES

The present disclosure is further illustrated by the following examples, which in no way should be construed as being limiting. That is, the specific features described in the following examples are merely illustrative and not limiting.

### Example 1

In Example 1, a hydrogel-coated medical device was formed by first soaking a 5×18 mm stent in 15% (wt/wt) aqueous poly(allylamine) solution to form a poly(allylamine) adhesive layer. Excess poly(allylamine) solution was wicked away with tissue paper, and the stent was allowed to dry. After drying, the solution left behind a hard poly(allylamine) film. The coated stent was then immersed in a hydrogel precursor solution for 5 minutes followed by a rinse with a PBS buffer. The resulting conformal hydrogel coating had a thickness of about 100 μm, and a strut width of about 100 μm.

### Example 2

In Example 2, a hydrogel-coated medical device was formed in a similar way as Example 1. However, in Example 2, the poly(allylamine) adhesive layer was formed by dipping the stent in a 5% (wt/wt) aqueous poly(allylamine) solution, wicking off excess moisture, and allowing it to dry in air.

### Example 3

In Example 3, a hydrogel-coated medical device was formed by first immersing a 4×15 mm coronary stent in a 2% (wt/wt) aqueous poly(allylamine) solution to form a poly(allylamine) adhesive layer on the stent. Excess poly(allylamine) solution was wicked away with tissue paper, and the stent was allowed to dry.

Next, a hydrogel precursor solution was formed according to Table 1.

TABLE 1

| | | |
|---|---|---|
| sample volume | 500.0 | uL |
| 8 arm PEG-NHS (solid) | 104 | mg |
| RGD-lysine (50 mg/ml in PBS) | 60 | uL |
| 1x PBS Buffer | 188 | uL |
| Cell in PBS | 200 | uL |
| PEG-diamine (400 mg/ml in PBS) | 52 | uL |

The 8-arm PEG-NHS had a molecular weight of 40 kg/mol, the PEG-diamine had a molecular weight of 2 kg/mol, and the solution contained 25% solids by weight. The molar ratio of amines to NHS groups was 1:1. Also added was 10 mM RGD-lysine to provide specific attachment sites for the human mesenchymal stem cells, 4 million of which were mixed into 0.5 mL of the hydrogel precursor solution. The RGD-lysine served as a capping group that reacted with the ends of the 8-arm PEG-NHS rather than allowing the 8-arm PEG-NHS to polymerize with the PEG-diamine. The human mesenchymal stem cells also disrupt crosslinking because they produce a plethora of factors with primary amines that cap the PEG-NHS groups in a similar manner. As in Examples 1 and 2, the coated stent was then immersed in the hydrogel precursor solution and rinsed with a PBS buffer.

### Exemplary Embodiments

Certain embodiments according to the present invention provide a method for forming medical devices conformally coated with a hydrogel having a wide variety of therapeutic uses. For instance, this method provides a platform for stem cells to accomplish one or more of the following: release paracrine factors that promote regeneration in damaged tissue, prevent further tissue damage, and recruit endogenous stem cells to accelerate the healing process. In one aspect, according to certain embodiments of the present invention, the method for forming a hydrogel-coated medical device includes immersing a medical device in a polymer solution to form an adhesive layer on an outer surface of the medical device, drying the medical device, contacting the medical device with a hydrogel precursor solution having a pH of less than 7 to react the adhesive layer with the hydrogel precursor solution and form a conformal hydrogel coating, and rinsing away excess hydrogel precursor solution from the medical device to form a uniform conformal hydrogel coating. In certain embodiments, the adhesive layer comprises at least one polymer having at least one amine group, pH-modifying abilities, and reactivity with an activated ester.

In accordance with certain embodiments of the present invention, the hydrogel precursor solution is formed by mixing a first neutral water-soluble polymer that forms hydrogels when crosslinked in water with at least two activated ester groups and mixing a second neutral water-soluble polymer that forms hydrogel when crosslinked in water with at least two amine groups to form a hydrogel polymer network. In some embodiments, the first and second neutral water-soluble polymers that form hydrogels when crosslinked in water are PEG. According to certain embodiments, the hydrogel precursor solution has a pH of about 6. In such embodiments, the adhesive layer adjusts the pH of the hydrogel precursor solution on contact with the medical device from about 6 to about 7.4. In certain embodiments, the hydrogel precursor solution comprises a plurality of stem cells. In such embodiments, the hydrogel precursor solution comprises RGD oligopeptide adhesion molecules. As such, according to some embodiments, the conformal hydrogel coating forms an anti-fouling surface on the medical device.

In accordance with certain embodiments of the present invention, the adhesive layer comprises at least one of a poly(allylamine), a polylysine, a polyethyleneimine, or a silicone. In certain embodiments, the adhesive layer comprises poly(allylamine). In other embodiments, the adhesive layer comprises silicone.

In accordance with certain embodiments of the present invention, immersing the medical device in the hydrogel precursor solution comprises step-growth polymerizations. In such embodiments, the step-growth polymerizations comprise reacting PEG with one of NHS ester/amine, isocyanate/amine, epoxy/amine, isothiocyanate/amine, alcohol/glutamate, thiol/maleimide, isocyanate/alcohol, or isocyanate/polyol reaction chemistries. According to certain embodiments, the step-growth polymerizations comprise reacting PEG with NHS ester/amine. In other embodiments, the step-growth polymerizations comprise reacting PEG with isocyanate/alcohol or isocyanate/polyol. In such embodiments, the method further comprises embedding a non-toxic catalyst in the hydrogel precursor solution.

In accordance with certain embodiments of the present invention, the medical device comprises any insertable medical device. In certain embodiments, the medical device comprises a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor. In some embodiments, the medical device is a stent. In such embodiments, the adhesive layer comprises a poly(allylamine). In other embodiments, the medical device is a pacemaker electrode. In such embodiments, the adhesive layer comprises a poly(allylamine) coating applied to silicone.

In another aspect, according to certain embodiments of the present invention, a method for forming a hydrogel-coated medical device includes immersing a medical device in a polymer solution to form an adhesive layer on an outer surface of the medical device and contacting the medical device with a hydrogel precursor solution having only monovalent cations to bond the adhesive layer with the hydrogel precursor solution and form a conformal hydrogel coating. In certain embodiments, the adhesive layer comprises at least one polymer having at least three positively charged pendant groups and a water-soluble multivalent cation that leaches into the adjacent aqueous solution.

In accordance with certain embodiments of the present invention, the hydrogel precursor solution is formed by mixing a first water-soluble polymer that forms hydrogels when crosslinked in water with at least three negatively charged pendant groups and mixing a second water-soluble cation with a valency of at least two that forms hydrogels when mixed with polyanions in water. In some embodiments, the first water-soluble polymer that forms hydrogels when crosslinked in water comprises sodium alginate.

In accordance with certain embodiments of the present invention, the adhesive layer forms multiple ionic bonds with the polyanions. In certain embodiments, the adhesive layer leaches multivalent cations into the hydrogel precursor solution on contact with the medical device to a final concentration of at least 0.01 mM.

In accordance with certain embodiments of the present invention, contacting the medical device with the hydrogel precursor solution comprises ionic cross-linking. In certain embodiments, the ionic cross-linking comprises mixing a negatively charged polyelectrolyte and a multivalent cation. In such embodiments, the negatively charged polyelectrolyte comprises sodium alginate, sodium hyaluronate, poly(acrylic acid) sodium salt, poly(methacrylic acid) sodium salt, or poly(styrene sulfonate) sodium salt. Additionally, in such embodiments, the multivalent cation comprises Ca²⁺, Al³⁺, Fe³⁺, or Cu²⁺. In certain embodiments, the ionic cross-linking comprises mixing sodium alginate with CaCl₂. In other embodiments, the ionic cross-linking comprises mixing sodium hyaluronate with CaCl₂.

In another aspect, certain embodiments according to the present invention provide a hydrogel-coated medical device having a wide variety of therapeutic uses. For instance, the hydrogel-coated medical device provides a platform for stem cells to accomplish one or more of the following: release paracrine factors that promote regeneration in damaged tissue, prevent further tissue damage, and recruit endogenous stem cells to accelerate the healing process. According to certain embodiments, the hydrogel-coated medical device comprises a medical device and a conformal hydrogel coating deposited on an outer surface of the medical device. In such embodiments, the conformal hydrogel coating comprises a PEG hydrogel, a plurality of stem cells, and RGD oligopeptide adhesion molecules. In certain embodiments, the conformal hydrogel coating immobilizes the stem cells in the hydrogel but permits permeation of nutrients, waste, and growth factors.

In accordance with certain embodiments of the present invention, the medical device may comprise any insertable medical device. In such embodiments, the medical device may comprise a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor. In certain embodiments, for example, the medical device may comprise a stent. According to certain embodiments, for instance, the medical device may comprise a stainless steel stent. In further embodiments, for example, the medical device may comprise a pacemaker electrode.

According to certain embodiments of the present invention, the hydrogel-coated medical device is formed by immersing a medical device in a polymer solution to form an adhesive layer on an outer surface of the medical device, drying the medical device, contacting the medical device with a hydrogel precursor solution having a pH of less than 7 to react the adhesive layer with the hydrogel precursor solution and form a conformal hydrogel coating, and rinsing away excess hydrogel precursor solution from the medical device to form a uniform conformal hydrogel coating. In certain embodiments, the adhesive layer comprises at least one polymer having at least one amine group, pH-modifying abilities, and reactivity with an activated ester.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A method for forming a hydrogel-coated medical device, comprising:
(a) immersing a medical device in a polymer solution to form an adhesive layer on an outer surface of the medical device, wherein the adhesive layer comprises at least one polymer having:
i. at least one amine group;
ii. pH-modifying abilities; and
iii. reactivity with an activated ester; and
(b) contacting the medical device with a hydrogel precursor solution having a pH of less than 7 to react the adhesive layer with the hydrogel precursor solution and form a conformal hydrogel coating.

2. The method according to claim 1, wherein
the hydrogel precursor solution is formed by mixing a first neutral water-soluble polymer that forms hydrogels when crosslinked in water with at least two activated ester groups and mixing a second neutral water-soluble polymer that forms hydrogels when crosslinked in water with at least two amine groups to form a hydrogel polymer network, and
the first, second, or both neutral water-soluble polymers that form hydrogels when crosslinked in water comprise polyethylene glycol (PEG).

3. The method according to claim 1,
wherein the hydrogel precursor solution has a pH of about 6 or below, and
the adhesive layer adjusts the pH of the hydrogel precursor solution on contact with the medical device to at least about 7.4.

4. The method according to claim 1, wherein the hydrogel precursor solution comprises a plurality of stem cells.

5. The method according to claim 4, wherein the hydrogel precursor solution comprises Arg-Gly-Asp (RGD) oligopeptide adhesion molecules.

6. The method according to claim 1, wherein the conformal hydrogel coating forms an anti-fouling surface on the medical device.

7. The method according to claim 1, wherein the adhesive layer comprises at least one of a poly(allylamine), a polylysine, or a polyethylenimine.

8. The method according to claim 7, wherein the adhesive layer comprises poly(allylamine).

9. The method according to claim 1, wherein contacting the medical device with the hydrogel precursor solution comprises step-growth polymerizations,
wherein the step-growth polymerizations comprise reacting PEG with one of N-hydroxysuccinimide (NHS) ester/amine, isocyanate/amine, epoxy/amine, isothiocyanate/amine, alcohol/glutamate, thiol/maleimide, isocyanate/alcohol, or isocyanate/polyol reaction chemistries.

10. The method according to claim 9, wherein the step-growth polymerizations comprise reacting PEG with NHS ester/amine.

11. The method according to claim 9, wherein the step-growth polymerizations comprise reacting PEG with isocyanate/alcohol or isocyanate/polyol.

12. The method according to claim 11, further comprising embedding a non-toxic catalyst in the hydrogel precursor solution.

13. The method according to claim 1, wherein the medical device comprises a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor.

14. A method for forming a hydrogel-coated medical device, comprising:
(a) immersing a medical device in a polymer solution to form an adhesive layer on an outer surface of the medical device, wherein the adhesive layer comprises at least one polymer having:
i. at least three positively charged pendant groups; and
ii. a water-soluble multivalent cation that leaches into the adjacent aqueous solution; and
(b) contacting the medical device with a hydrogel precursor solution having only monovalent cations to bond the adhesive layer with the hydrogel precursor solution and form a conformal hydrogel coating.

15. The method according to claim 14, wherein
the hydrogel precursor solution is formed by mixing a first water-soluble polymer that forms hydrogels when crosslinked in water with at least three negatively charged pendant groups and mixing a second water-soluble cation with a valency of at least two that forms hydrogels when mixed with polyanions in water,
the first water-soluble polymer that forms hydrogels when crosslinked in water comprises sodium alginate, and
the adhesive layer forms multiple ionic bonds with the polyanions.

16. The method according to claim 15, wherein
the adhesive layer leaches multivalent cations into the hydrogel precursor solution on contact with the medical device to a final concentration of at least 0.01 mM, and
contacting the medical device with the hydrogel precursor solution comprises ionic cross-linking comprising mixing a negatively charged polyelectrolyte and a multivalent cation,
the negatively charged polyelectrolyte comprises sodium alginate, sodium hyaluronate, poly(acrylic acid) sodium salt, poly(methacrylic acid) sodium salt, or poly(styrene sulfonate) sodium salt, and
the multivalent cation comprises Ca²⁺, Al³⁺, Fe³⁺, or Cu²⁺.

17. The method according to claim 16, wherein the ionic cross-linking comprises one of mixing sodium alginate with CaCl₂ and mixing sodium hyaluronate with CaCl₂

18. A hydrogel-coated medical device, comprising:
(a) a medical device; and
(b) a conformal hydrogel coating deposited on an outer surface of the medical device, wherein the conformal hydrogel coating comprises a PEG hydrogel, a plurality of stem cells, and Arg-Gly-Asp (RGD) oligopeptide adhesion molecules.

19. The hydrogel-coated medical device according to claim 18, wherein the conformal hydrogel coating immobilizes the stem cells in the hydrogel but permits permeation of nutrients, waste, and growth factors.

20. The hydrogel-coated medical device according to claim 18, wherein the medical device comprises a stent, a stent sleeve, a pacemaker, an implantable cardioverter-defibrillator, a pacemaker electrode, an implantable cardioverter-defibrillator lead, a biventricular implantable cardioverter-defibrillator lead, an artificial heart, an artificial valve, a ventricular assist device, a balloon pump, a catheter, a central venous line, an implant, or a sensor.
